(19) United States
(12) Patent Application Publication
(10) Pub. No.: US 2004/0059096 A1
(43) Pub. Date: Mar. 25, 2004

(54) Anti-ciguatoxin monoclonal antibody

(76) Inventors: Masahiro Hirama, Miyagi (JP); Hiroki Oguri, Miyagi (JP); Ikuo Fujii, Osaka (JP); Takeshi Tsumuraya, Osaka (JP)

Correspondence Address: BURNS DOANE SWECKER & MATHIS L L P, POST OFFICE BOX 1404, ALEXANDRIA, VA, 22313-1404, US

(21) Appl. No.: 10/470,010

(22) PCT Filed: Feb. 1, 2002
(86) PCT No.: PCT/JP02/00863

(30) Foreign Application Priority Data
Aug. 16, 2001 (JP) 2001247120

(51) Int. Cl. G01N 33/53; C07K 16/18; C07K 14/47

(52) U.S. Cl. 530/388.1; 530/403; 435/7.1

(57) ABSTRACT

The present invention offers the monoclonal antibodies that have a high specificity to ciguatoxins, which are the causative poisons of the ciguatera poisoning. The monoclonal antibodies were produced using synthetic hapten conjugates as artificial antigens, which were prepared by the conjugation of a synthesized hapten consisting of the IJKLM-ring fragment of ciguatoxins with proteins such as KLH, BSA, etc. The monoclonal antibodies show a specific reactivity to ciguatoxins, but little or almost no reactivity to other marine polyether toxins such as the okadaic acid, etc.

### TECHNICAL FIELD

The present invention relates to monoclonal antibodies produced to ciguatoxins which are the main causative toxins in the food poisoning ciguatera, and more particularly to antibodies produced by using a synthetic hapten having the partial structure of particular type of ciguatoxins.

### PRIOR ART

The food poisoning ciguatera frequently occurs in wide coral reef sea areas such as Polynesia, Hawaii, Okinawa, The Caribbean Sea etc., and affects more than 20,000 people annually (YasuOizumi, Editorial translation, “Dangerous marine organisms (Resources for drug discovery in the 21th century)” p144, Hirokawa Book Store and Yoshiro Hashimoto Ed., “Poisons in fishes and shellfishes” p96-97, Institute Publication Center). The ciguatera toxins are produced by phytoplanktons and transferred to many fishes and shellfishes throughout the food-chain. Differing from tetrodotoxin, it causes serious social problems for humans because it causes wide toxication of the many esculent fishes, which are the basis of fishery. The following symptoms usually occur as the result of poisoning: paralysis, diarrhea, vomiting, metallic taste, dryness of the mouth, abdominal spasm, headache, muscular pain, nervousness, vertigo, cyanosis, insomnia, extreme fatigue and weakness, abasia, etc. Also sometimes a strong pain in the arm and foot, a feeling like loosing of the teeth, pain of the back of the eyes, dysopia, skin disorders including erythema, measles, swelling, a feeling of prickliness, etc., and hair and nail loss. Sometimes an inversion in the sensation of cold and warm as a person feels cold to a warm substance is occurred. When touching a cold substance, a person may feel as though they are touching dry ice or a person may experience an electroshock. In serious cases, a person experiences a large muscular movement regulation abnormality, paralysis and convulsion, they then fall into a coma and finally die. Recovery from such symptoms is very slow, and takes several months to several years. As a result, fishermen do not go out fishing.

Ciguatoxins, which are the main causative poison of the food poisoning ciguatera, are not a single chemical compound but mixtures of many toxic substances. The relative structure of a ciguatoxin (CTX1B) was clarified in 1989 (Murata, M. et. al, J. Am. Chem. Soc. 1989, 111, 8929; Murata, M. et. al, J. Am. Chem. Soc. 1990, 112, 4380). In addition, its absolute configuration was also clarified (Satake, M. et. al, J. Am. Chem. Soc. 1997, 119, 11325; Oguri, H. et. al, Tetrahedron 1997, 53, 3057). To date, many homologues are known in ciguatoxins and 4 species of CTX (LD50=0.35 μg/kg), 54-deoxy-CTX, CTX3C (LD50=1.3 μg/kg) and 51-oxy-CTX3C (LD50=0.27 μg/kg) are major toxins among them.

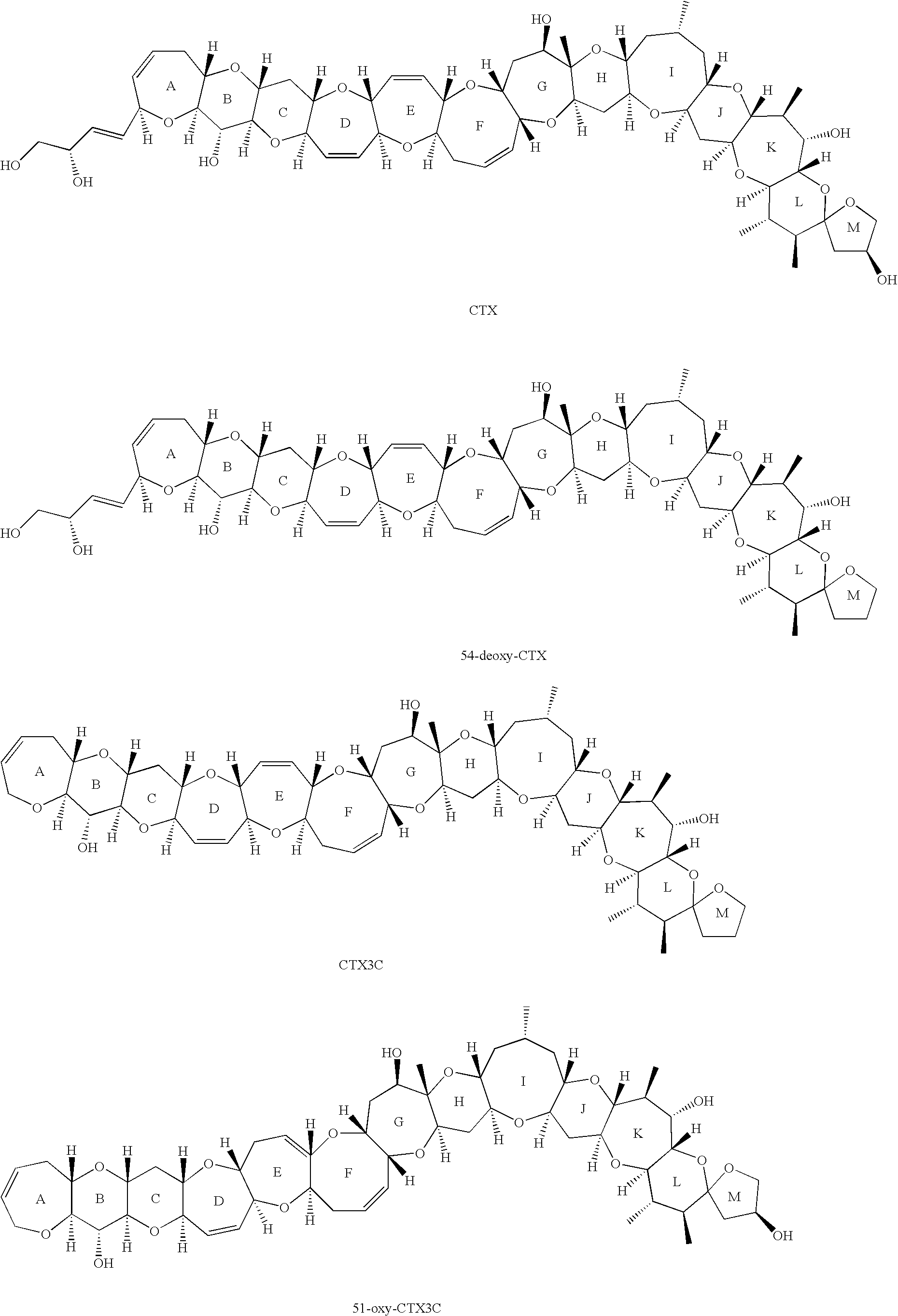

These ciguatoxins (ciguatera poisons) can only be collected in a trace amounts from nature (only 0.35 mg of ciguatoxins can be obtained from 4 t of 850 moray eels) and are difficult to produce by fermentation. Therefore, a problem arises in the preparation of the antibodies to them. It has been reported that it is possible to prepare a monoclonal antibody by immunization of mice with an antigen of the conjugate, prepared by the coupling of the ciguatoxins with human serum albumin by the carbodiimide method (Toxicon (1997), vol.15, pp.317-325). This antibody binds to the 1 ng/mL ciguatoxin, but shows a cross activity with the 5 ng/mL okadaic acid, and the difference in these affinities is only about 5 times (Journal of Clinical Laboratory Analysis Vol. 6, page 54, 1992). It has also been shown that it demonstrates a cross activity with brevetoxins, maitotoxin, palytoxin, etc., (Journal of AOAC International, 1988, 81, 727-735) but no detailed data was published. From the viewpoint of such results, this method is not considered to be practical as a reliable assay method with a high selectivity.

Furthermore, U.S. Pat. No. 4,816,392 discloses a method for simply detecting ciguatoxins and other polyether toxins from the tissues of the fishes contaminated with the ciguatera poisons. This method is also similarly based on the enzyme immunoassay method along with aforementioned one. And, another method for quickly extracting the ciguatoxins using a monoclonal antibody, which react with antigen determinants specific to ciguatoxins, has been disclosed (Japanese Patent Disclosure (TOKUHYO) Hei 8-500433). However, both of these methods also have the aforementioned problems since the ciguatoxins extracted from the fishes contaminated with the ciguatera poison are used for the antibody production.

The inventors of the present invention previously prepared 3 monoclonal antibodies using protein conjugates coupled with a synthetic right ABC ring of ciguatoxins as a hapten, but these antibodies showed only a very weak affinity (Synthesis 1999, No. SI, 1431-1436). Although immunization using a synthetic hapten (JKLM ring fragment) has also been tried, it has not produced any monoclonal antibodies (Toxicon (2000), vol.38, pp.669).

Problem to be Resolved by the Invention

Thus, the development of a highly sensitive assay method for ciguatoxins and monoclonal antibodies with a high specificity to the toxins, which are causative toxins causing a serious poisoning, has been urgently awaited.

Means for Settlement of the Problems

In the present invention, when non-protein toxins etc. (natural organic compounds) are difficult to obtain, the partial structures to become antigenic determinants were synthesized, and the methods of preparing a monoclonal antibody specific to the toxin's main body using these synthetic haptens, were applied to ciguatoxins. Namely, a hapten consisting of the IJKLM-ring fragment of ciguatoxins was synthesized and the protein conjugates were prepared by coupling the proteins such as KLH, BSA, etc. with the synthetic hapten. A monoclonal antibody with a high specificity to ciguatoxins, the main causative toxins of the food poisoning ciguatera, was produced by immunization of mammals using the conjugate of the synthetic hapten as an artificial antigen and by cloning of the infused cells of mammalian lymphocytes and myeloma cells. The monoclonal antibody produced in this manner showed a specific reactivity to ciguatoxins and little or no reactivity to other marine polyether toxins such as okadaic acid.

Namely, the present invention is a monoclonal antibody which specifically binds to ciguatoxins.

The present invention is also a monoclonal antibody specific to ciguatoxins which specifically binds to a protein conjugate prepared by coupling a synthetic hapten, having a partial structure capable of being an antigenic determinant for ciguatoxins, and a protein.

The monoclonal antibody specifically recognize the ciguatoxins (cf. the chemical structure formula above) which contain the IJKLM-ring fragment as an antigenic determinant. That is to say, though the monoclonal antibodies of the present invention specifically react with ciguatoxins, the reactivity for the marine polyether toxins except for ciguatoxins is low.

The examples of the marine polyether toxins except for ciguatoxins includes those having a chemical formula similar to that of ciguatoxins, ex. okadaic acid

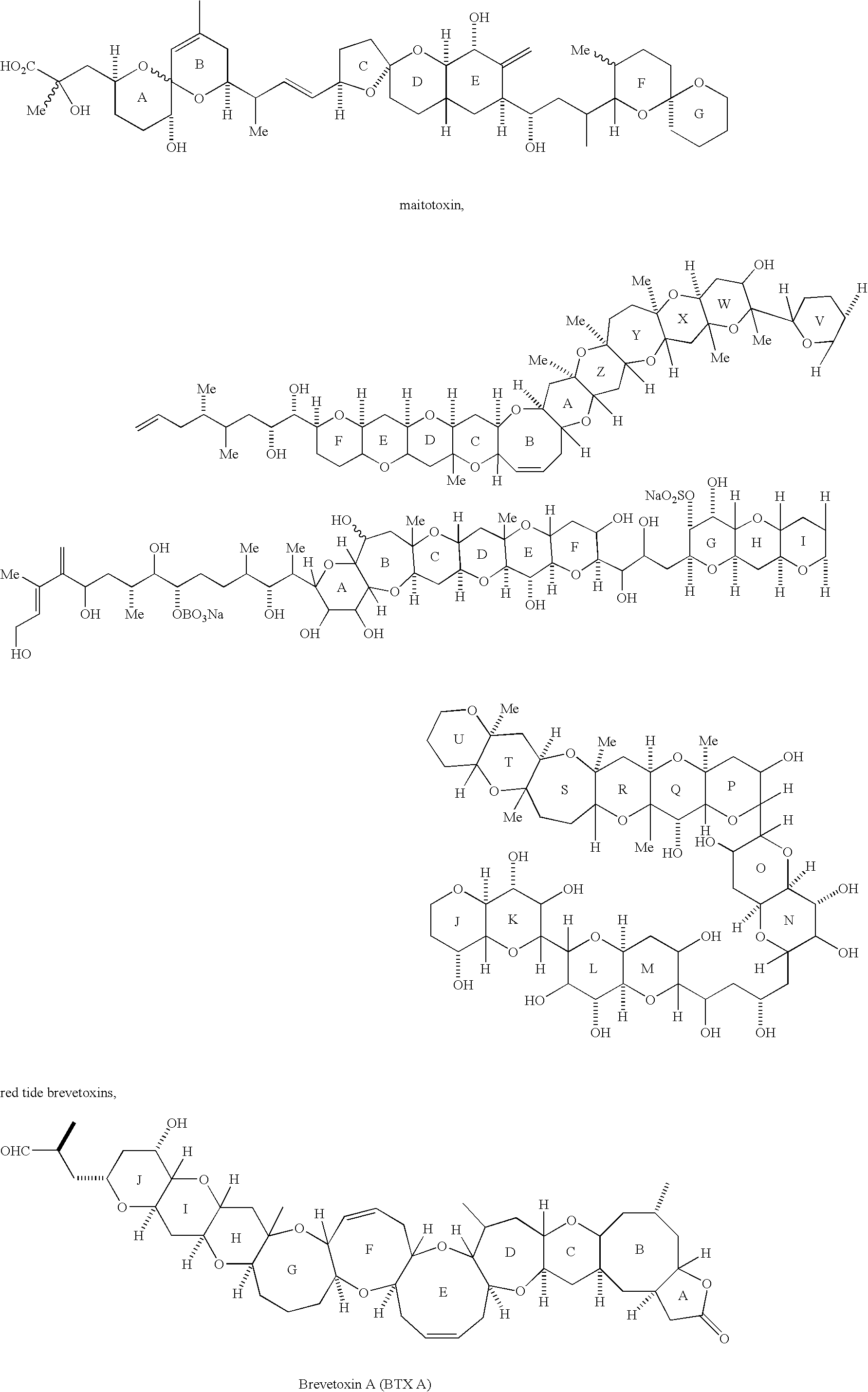

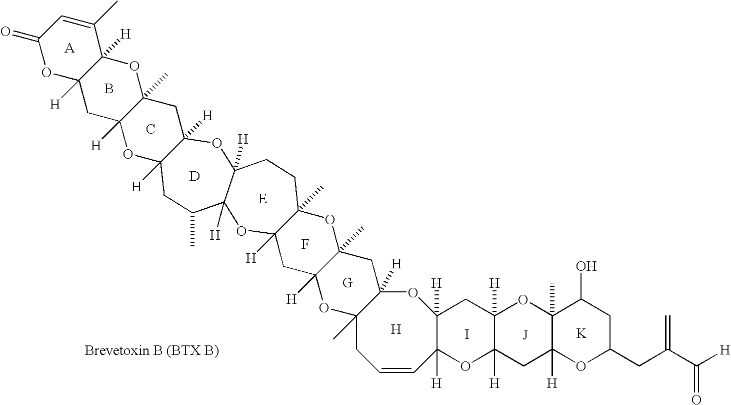

The low reactivity to these marine polyether toxins means that the reactivity to these toxins are extremely lower than the reactivity to ciguatoxins, and more specifically, the dissociation constants to these toxins are lower by 2 digits (lower than 100 times) than the dissociation constants to ciguatoxins (the highest constants among various ciguatoxins).

One of the important features of the present invention is that the synthetic hapten used here is not natural extract but is artificially synthesized. Also, it is preferred that the protein conjugates synthesized using the synthetic hapten are protein conjugates represented by the following chemical formula:

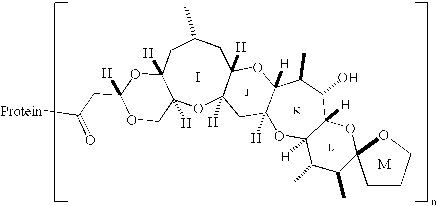

Furthermore, the present invention includes the detection reagents for ciguatoxins, comprising any of these monoclonal antibodies as an active component.

In addition, the present invention includes the protein conjugates by coupling the synthetic haptens, having a partial structure capable of being an antigen determinant of ciguatoxins, and a protein, and preferably the protein conjugates represented by the following chemical formula (wherein n is a positive number).

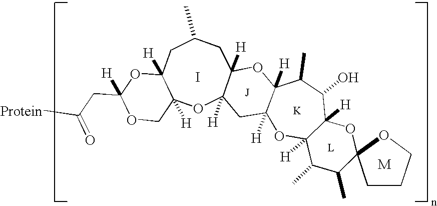

### BRIEF DESCRIPTION OF THE FIGURES

FIG. 1 shows the flow of the synthesis of the IJKLM-ring fragment hapten and its protein conjugates (1).

FIG. 2 shows the flow of the synthesis of the IJKLM-ring fragment hapten and its protein conjugates (2).

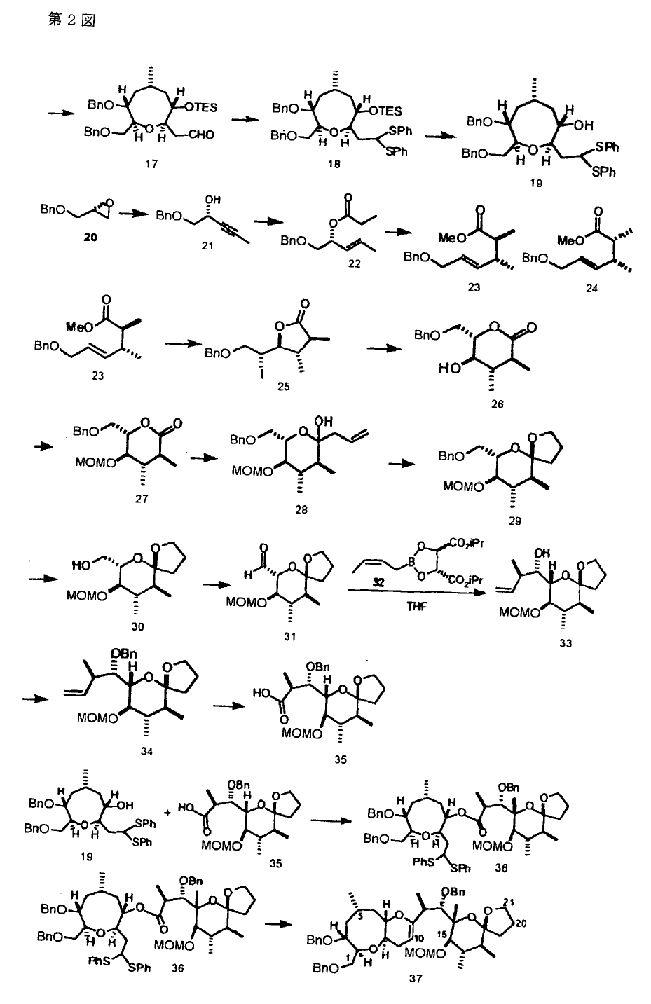

FIG. 3 shows the flow of the synthesis of the IJKLM-ring fragment hapten and its protein conjugates (3).

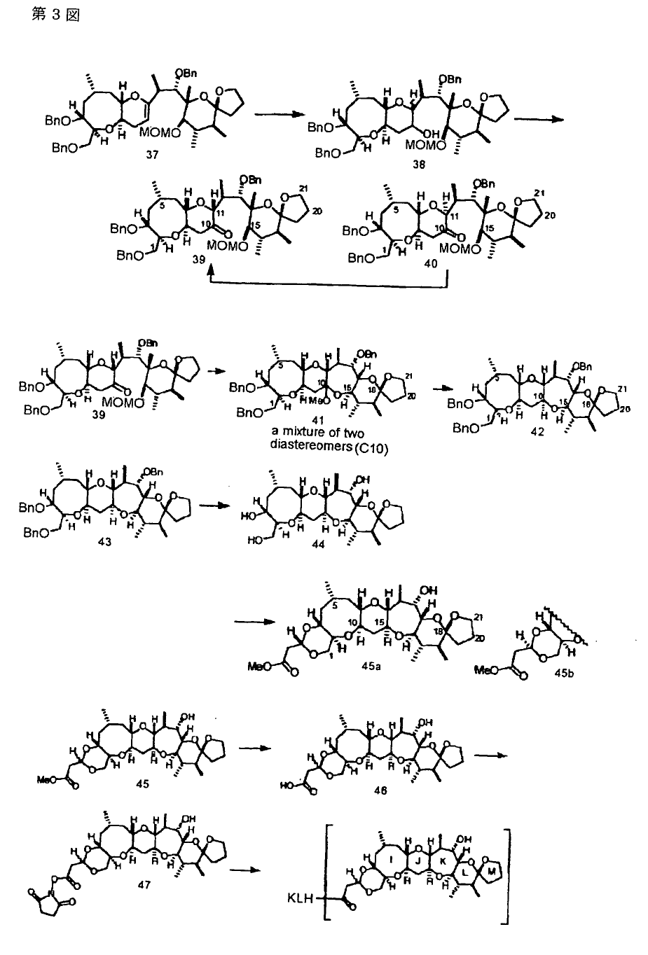

FIG. 4 shows the result of a competitive inhibition experiment of monoclonal antibody 3D11 using ciguatoxin CTX3C.

FIG. 5 shows the Klotz plotting for calculating the connectivity (dissociation constant: Kd) of monoclonal antibody 3D11 for ciguatoxin CTX3C.

### EMBODIMENTS OF THE INVENTION

Antibody preparation using the natural ciguatera toxins is extremely difficult, because the procurement of the natural ciguatera toxins is very difficult. In addition, it is difficult to specify which part of the natural toxins become an antigenic determinant, especially when the ciguatera toxins which have enormous and complicated molecular structures of 3 nanometers are used, because it is known that the size of the antigens to which the antibodies (immunoglobulins) bind is generally around 7˜9 amino acid residues. With the present invention, the IJKLM-ring fragment,

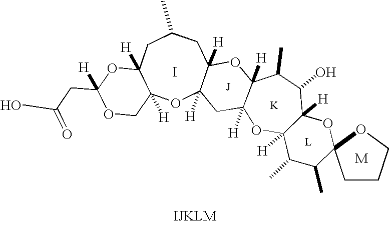

which is a partial structure of the terminal of the ciguatoxins, was designed as a hapten, and was artificially synthesized. The protein conjugates of this synthetic hapten were used for immunization of mice to obtain the monoclonal antibodies, which react to the hapten part of the ciguatoxins as an antigenic determinant. Since low-molecular haptens themselves are low in antigenicity, the conjugates coupled with carrier proteins are generally used as immunogens. And, if a toxin's main body is used for conjugation, it is likely to fatally damage any animals immunized with the conjugates, because ciguatoxins are deadly poisons to mammals. Like the present invention, this problem can be solved if nontoxic partial structures of toxins are used as haptens.

The preparation method of the conjugates, by coupling haptens to proteins, is important for the successful preparation of the monoclonal antibodies, which react, with an antigenic determinant of toxin's main body using synthetic partial structures. In the conventional anti-ciguatoxin antibody preparation, conjugates which coupled ciguatoxins (1 μg) with human serum albumin (HSA 1 mg) using the carbodiimide method are produced (Toxicon (1997), vol.15, pp.317-325). Generally, the carbodiimide method is effective in order to couple the lysine residues of serum albumin, if the low molecule (hapten) part is carboxylic acid. However, in the case of the above mentioned literature, there is a question involving the steps which produce conjugates with the serum albumin because ciguatoxins do not contain carboxylic acid as a functional group. As a possibility, it can be assumed that the hydroxyl group on the ciguatoxin side and the carboxylic acid on the albumin side are coupled in an ester bond, but it is difficult to efficiently form such ester-bond in the presence of the many active lysine residues that exist in albumin. In addition, it is thought that the conjugates which are combined with the ester are hydrolyzed by lipase, etc., possibly causing a problem with its stability during immunization in vivo.

With the present invention, the junction of the haptens and proteins was designed in order to expose hapten fragments as the antigenic determinant with an adequate length from the surface of the proteins. Proteins and haptens may be coupled by any common methods. For example, proteins and hapten fragments may be coupled by the coupling of the haptens containing carboxylic acids or aldehydes with the amino group of ricin in the proteins, or by the coupling of the haptens containing maleinimides with the thiol groups of cystein in the proteins. With the present invention, in particular, when using the carboxylic acids of the hapten terminals, we succeeded in efficiently producing the conjugates shown in the formula below, in which the amide bond was stable.

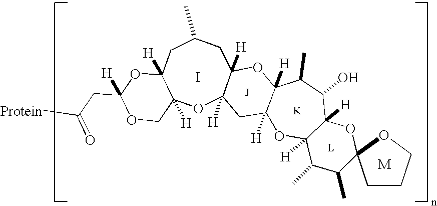

Here, the carrier proteins which have immunogenicity are adequate, and any proteins which are adequate for this purpose may be used. It is especially possible to use BSA (bovine serum albumin) or KLH (keyhole limpet hemocyanin). In the formula, “n” is a positive integer which is determined by the type of proteins used. When using BSA, “n”'s value will be somewhere between 10˜20, and when using KLH, it's value will be somewhere between 80˜200.

Next, the monoclonal antibodies which react with these hapten parts to become antigenic determinants were prepared by routine procedures of immunizing mice using the protein conjugates produced by the above process.

The present invention is illustrated in more detail, but it is not intended to limit the scope of the present invention.

### EXAMPLES

1. Synthesis of the IJKLM Ring Fragment Hapten and its Protein Conjugates.

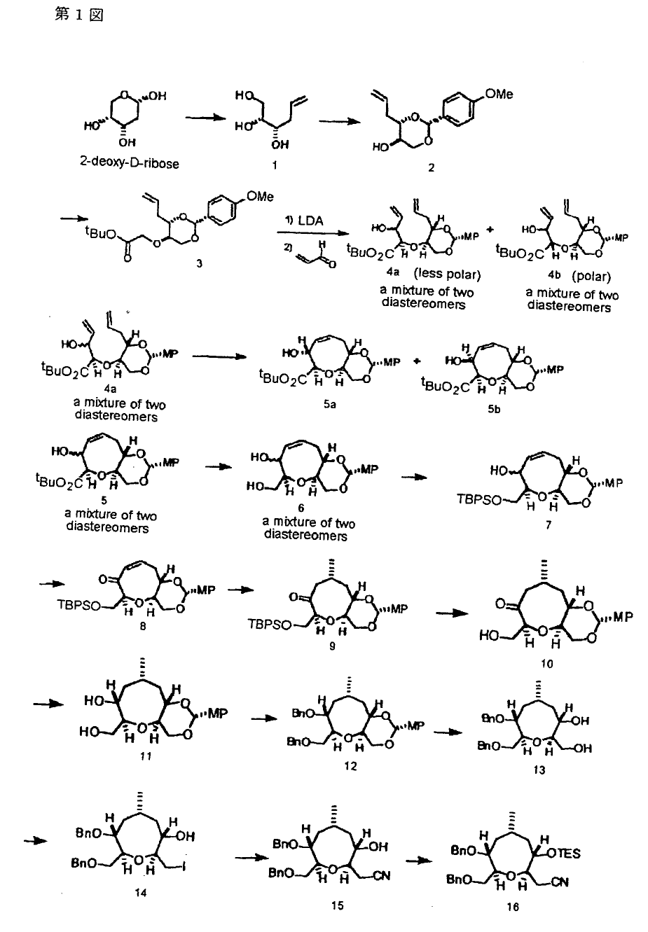

The flow charts of these synthesis are shown in FIGS. 1˜3. The numbers in the parenthesis appended to the chemical compounds in the sentences correspond to the chemical compound numbers in these figures.

Ph₃P⁺CH₂Br⁻ (167 g, 466 mmol) and THF (2000 mL) were put in a 3 L three mouth flask and tBuOK (50.2 g, 447 mmol) was added to it while stirring at 0° C. After stirring for 1 hour, 2-deoxy-D-ribose (25.0 g, 186 mmol) was added and then it was further stirred for 48 hours at room temperature. After the confirmation of elimination of raw materials by TLC, the solvents were eliminated by filtration using the Buechner funnel, and then, white crystals (1) (23.5 g, 178 mmol, 95%) were obtained by purification by silica gel column chromatography.

The crystals (1) (8.62 g, 65 mmol), p-MeOC₆H₄CH(OMe)₂ (17.8 g, 97.9 mmol), CH₂Cl₂ (200 mL) and CSA (3.03 g, 13.0 mmol) were put in a 300 mL eggplant-type flask, and it was refluxed at 45° C. for 5 days. After the completion of the reaction was confirmed by NMR, Et₃N (18 mL) was added, and the reaction was stopped. After the elimination of the solvents, it was purified by silica gel column chromatography, and white crystals (2) (13.33 g, 53.3 mmol, 87%) were obtained.

Under the argon atmosphere, the crystals (2) (12.0 g, 47.9 mmol), THF (160 mL), DMF (16 mL) and BrCH₂CO₂tBu (9.2 mL, 62 mmol) were put in a 300 mL eggplant-type flask, and NaH (60% in mineral oil: 2.3 g, 58 mmol) was added while stirring at 0° C. After stirring for 18 hours at room temperature, saturated sodium hydrogen carbonate aqueous solution was added. It was extracted with diethyl ether, and dried with sulfuric anhydride magnesium, after the organic layer was washed with saturated sodium hydrogen carbonate aqueous solution and saturated salt solution. After the elimination of the solvents, it was purified by silica gel column chromatography, and white crystals (3) (14.3 g, 39.2 mmol, 82%) were obtained.

Under the argon atmosphere, THF(19 mL) and diisopropylamine (0.8 mL, 5.75 mmol) were put in a 100 mL 2-mouth flask, and after cooling to −78° C., n-BuLi (2.95 mL, 4.61 mmol) was slowly dropped. It was warmed to 0° C., and then cooled to −78° C. again. The ester crystals (3) (1.4 g, 3,84 mmol) were slowly dropped in the flask. Distilled acrolein (0.27 mL, 4.05 mmol) was further added, and it was stirred for 10 minutes. After the reaction was completed, it was extracted with diethyl ether, the organic layer was washed with saturated ammonium chloride aqueous solution and saturated salt solution, and then dried with sulfuric anhydride magnesium. After the elimination of the solvents, it was purified by silica gel column chromatography, and further separated to two fractions giving colorless oily substances (4a, 4b) with yields of 47% (760 mg, 1.80 mmol) and 30% (484 mg, 1.15 mmol), respectively.

Under the argon atmosphere, the substance (4a) (2.9 g, 6.9 mmol) and CH₂Cl₂ (700 mL) were put in a 1L 3-mouth flask, the Grubbs catalyst (284 mg, 345 μmol) was added, and it was refluxed at 40° C. for 2 days. After the elimination of the raw materials was confirmed by TLC, Et₃N (20 mL) was added, and the reaction was stopped. After the elimination of the solvents, it was purified by silica gel column chromatography and white crystals (5a, 5b) (2.0 g, 5.1 mmol, 74%) were obtained.

Under the argon atmosphere, LiAlH₄ (285 mg, 7.52 mmol) and THF (0.5 mL) were put in a 100 mL eggplant-type flask, and the diastereomer mixture (5) (1.97 g, 5.02 mmol) dissolved in THF (10 mL) was dropped while stirring at 0° C. It was warmed to the room temperature, and further stirred for 30 minutes. After the elimination of the raw materials was confirmed by TLC, water (290 μl), 15% sodium hydroxide aqueous solution (290 μl) and water (870 μl) were added in that order. After the filtration of the precipitates with celite and the elimination of the solvents, it was purified by silica gel column chromatography, and a white crystal mixture (6) (1.42 g, 4.42 mmol, 88%) containing 2 kinds of diastereomers was obtained.

Under the argon atmosphere, the crystals (6) (520 mg, 1.61 mmol), CH₂Cl₂ (15 mL), Et₃N (0.67 mL, 4.81 mmol), and DMAP (20 mg, 0.16 mmol) were put in a 50 mL eggplant-type flask. Subsequently, TBPSCl (0.57 mL, 3.3 mmol) dissolved in CH₂Cl₂ (7 mL) was slowly dropped, and it was stirred for 24 hours at room temperature. After the elimination of the raw materials was confirmed by TLC, saturated sodium hydrogen carbonate aqueous solution was added. It was extracted with diethyl ether, and the organic layer was washed with saturated salt solution. After it was dried with sulfuric anhydride magnesium and eliminated the solvents, it was purified by silica gel column chromatography, and an amorphous state substance (7) (902.7 mg, 1.61 mmol, >99%) of a mixture of 2 kinds of diastereomers was quantitatively obtained.

Under the argon atmosphere, CH₂Cl₂ (3.5 mL) and DMSO (0.51 mL, 7.2 mmol) were put in a 20 mL pear-type flask, and cooled to −78° C. (COCl)₂ (0.31 mL, 3.6 mmol) was dropped in the flask, and it was stirred for 20 minutes. Consecutively, the substance (7) (500 mg, 0.89 mmol) dissolved in CH₂Cl₂ (3.5 mL) was dropped, and further stirred for 30 minutes. After the elimination of the raw materials was confirmed by TLC, Et₃N (2.0 mL, 14 mmol) was added. Saturated sodium hydrogen carbonate was dropped, extraction was carried out with diethyl ether, and the organic layer was washed with saturated salt solution. It was dried with sulfuric anhydride magnesium, and the solvents were eliminated. An amorphous state substance (8) (394 mg, 0.71 mmol, 79%) was obtained by purification by phlorizil column chromatography.

Under the argon atmosphere, diethyl ether (14 mL) and CuCN (320 mg, 3.58 mmol) were put in a 50 mL 2-mouth flask, and it was cooled to −78° C., and dropped MeLi (1.0 M hexane solution 7.1 mL, 7.1 mmol ). It was warmed to 0° C. once, cooled to −78° C. again, and added the substance (8) (470 mg, 0.84 mmol). After stirring for 5 minutes, the elimination of the raw materials was confirmed by TLC, and then a few drops of ammonium chloride: ammonia (5:1) aqueous solution was added. In addition, it was added Et₃N (2.0 mL, 14 mmol), extracted with diethyl ether, and dried with sulfuric anhydride magnesium, after washing with ammonium chloride aqueous solution and saturated salt solution. After the elimination of the solvents, an amorphous state substance (9) (432 mg, 0.76 mmol, 90%) was obtained by purification by silica gel column chromatography.

The substance (9) (890 mg, 1.55 mmol) and THF (15.5 mL) was put in a 30 mL eggplant-type flask, and cooled to 0° C. A mixed the solvent of 1.0M TBAFin THF (2.33 mL, 2.33 mmol) and AcOH (0.133 mL, 2.33 mmol) was slowly dropped, and warmed to room temperature. After stirring for 20 hours, the solvents were eliminated. It was purified by silica gel column chromatography, and white crystals (10) (502 mg, 1.49 mmol, 96%) were obtained.

Under the argon atmosphere, NaBH(OAc)₃ (1.58 mg, 7.45 mmol) and CH₃CN (7 mL) were put in a 50 mL eggplant-type flask and cooled to −40° C. AcOH (1.5 mL, 26.2 mmol) was added, andnext, the crystals (10) (502 mg, 1.49 mmol) dissolved in CH₃CN (14 mL) was dropped. After stirring for 1.5 hours, a few drops of saturated ammonium chloride aqueous solution were added, a few drops of rochelle salt were further added and stirred for 1 hour. After it was dried with sulfuric anhydride magnesium, the solvents were eliminated, and it was purified by silica gel column chromatography, a chemical compound (11) (489 mg, 1.45 mmol, 97%) was obtained.

The chemical compound (11) (150 mg, 0.473 mmol), THF (2.5 mL) and DMF (0.5 mL) were put in a 50 mL eggplant-type flask, and it was stirred at 0° C. NaH (60% suspension in mineral oil: 114 mg, 2.84 mmol) was added, and after stirring for 10 minutes, benzil bromide (225 μL, 1.89 mmol) was dropped. After stirring for 18 hours at room temperature, water was dropped at 0° C., and the reaction was stopped. After it was diluted with ethyl acetate, the organic layer was extracted, and washed with saturated ammonium chloride aqueous solution and saturated salt solution, dried with sulfuric anhydride magnesium, filtered and concentrated. It was purified by silica gel chromatography, and a chemical compound (12) (261 mg, 0.503 mmol, >99%) was quantitatively obtained.

The compound (12) (200 mg, 0.386 mmol) was dissolved in methanol (4 mL); cation exchange resin (Dowex-20: 22 mg) was added and then the mixture was stirred for 12 hours at room temperature. After the reaction solution was diluted with methanol (8 mL), Et₃N (0.2 mL) was added, and the filtration was done with celite. The filtrate was concentrated and purified by silica gel chromatography, and a chemical compound (13) (138 mg, 0.343 mmol, 89%) was obtained.

Under the argon atmosphere, the chemical compound (13) (5.35 g, 0.394 mmol) and imidazole (1.83 g, 26.7 mmol) were dissolved in THF (70 mL). PPh₃ (4.22 g, 16.1 mmol) and I₂ (3.73 g, 14.7 mmol) were added in that order, and stirred for 20 hours. After the reaction solution was diluted with ethyl acetate, the organic layer was washed with saturated ammonium chloride aqueous solution and saturated salt solution, dried with sulfuric anhydride magnesium, filtered, and concentrated. It was purified by silica gel chromatography, and a chemical compound (14) (5.97 g, 11.7 mmol, 87%) was obtained.

The compound (14) (5.97 g, 11.7 mmol) was dissolved in DMSO(23 mL), added sodium cyanide(1.14 g, 23.4 mmol), then, heated to 40° C. and stirred for 16 hours. It was extracted with ethyl acetate, and washed with saturated salt solution. It was heated to 50° C., and stirred for 60 hours. After the reaction solution was diluted with ethyl acetate, the organic layer was washed 3 times with water, and washed again with saturated salt solution. After drying with sulfuric anhydride magnesium, filtration, concentration and purification by silica gel chromatography, a nitrile (15) (4.38 g, 10.7 mmol, 91%) was obtained.

Under the argon atmosphere, the nitrile (15) (4.38 g, 10.7 mmol), CH₂Cl₂ (23 mL) and 2.6-lutidine (2.04 mL, 17.6 mmol) were put in a 100 mL eggplant-type flask. It was cooled to −33° C., dropped slowly TESOTf (2.9 mL, 13 mmol), and stirred for 15 minutes. After the elimination of the raw materials was confirmed by TLC, saturated ammonium chloride aqueous solution was added, and the reaction was stopped. It was extracted with hexane-ethyl acetate, and the organic layer was washed with water, saturated ammonium chloride aqueous solution and saturated salt solution. It was dried with sulfuric anhydride magnesium, eliminated the solvents and purified by silica gel column chromatography, and a chemical compound (16) (4.78 g, 9.13 mmol, 85%) was obtained.

The chemical compound (16) (4.78 g, 9.13 mmol) was dissolved in CH₂Cl₂ (32 mL), and cooled to −78° C. Diisobutylaluminum hydride (0.94 M hexane solution 28.8 mL, 27.4 mmol) was dropped, and it was stirred for 40 minutes at −78° C. After the reaction solution was diluted with ethyl acetate, saturated rochelle salt solution (20 mL) and saturated ammonium chloride aqueous solution (10 mL) were added, and stirred intensively at room temperature for 1 hour. The organic layer was washed with saturated salt solution, dried with sulfuric anhydride magnesium, filtered and concentrated. It was purified by phlorizil column chromatography, and an aldehyde (17) was obtained. The aldehyde (17) was put in a 50 mL eggplant-type flask, PhSSPh (2.19 g, 10.0 mmol) and PBu₃ (3.00 mL, 12.0 mmol) were added. Since it caused a slight exothermia, after cooling to 0° C., it was stirred at room temperature for 12 hours. It was purified by silica gel chromatography [beforehand Et₃N (3 mL]-eluted and inactivated silica gel was used], and a chemical compound (18) (4.83 g, 6.33 mmol, 73%, (2 steps)) was obtained.

The chemical compound (18) (5.73 g, 7.86 mmol) was put in a 30 mL eggplant-type flask, and n-Bu₄NF (1.0 M THF solution 8.65 mL, 8.65 mmol) was slowly dropped. After stirring for 2 hours at room temperature, the solvents were eliminated, and a chemical compound (19) (4.61 g, 7.50 mmol, 95%) was obtainedbypurificationby silica gel column chromatography.

The chemical compound (20) (253 g, 1.54 mol, purchasable from Sigma-Aldrich), was dissolved in DMSO (770 mL) and put in a 3L round bottom flask. The solution was cooled with ice, lithium acetylide-ethylenediamine complex (173 g, 1.69 mol) was slowly added and it was stirred for 4 hours while keeping the temperature of the reaction solution at 15° C. Into this solution, t-BuOK (173 g, 1.54 mol) was added, and stirred for 13 hours at room temperature. After cooling with ice, 6N hydrochloric acid (total 1L) was gradually added, and it was neutralized to about pH8. The organic layer was washed with saturated salt solution, dried with sulfuric anhydride magnesium, filtered and concentrated. After purification by reduced-pressure distillation, a chemical compound (21) (230 g, 1.21 mol, 79%) was obtained.

LiAlH₄ (46 g, 1.2 mol) was added in a 3L round bottom flask. After cooling with ice, THF (450 mL) was added and the THF (150 mL) solution of the chemical compound (21) (229 g, 1.20 mol) was added while stirring well. After stirring for one day at room temperature, it was cooled with ice; then diethyl ether (1500 mL), water (10 mL), 15% sodium hydroxide solution (40 mL) and water (140 mL) were added in that order. The reaction solution was filtered using the Buechner funnel and the filtrate was concentrated and azeotroped 3 times with hexane. During the reactor vessel was cooled with ice, pyridine (300 mL) and propionyl chloride (115 mL, 1.32 mol) were added. One hour after stirring at room temperature, methanol (10 mL) was added. The reaction solution was concentrated and a chemical compound (22) (266 g, 1.07 mmol, 89%) was obtained by purification by silica gel column chromatography.

The chemical compound (22) (26.7 g, 108 mmol), THF (280 mL) and HMPA (140 mL) were put in a 1L round bottom flask, and cooled to −78° C. After addition of TMSCI (41.0 mL, 322 mmol), LDA [the THF solution (140 mL) prepared from Disoprorylamine (30.2 mL, 215 mmol) and n-BuLi (1.56 M in hexane; 103 mL, 161 mmol)] was dropped and naturally warmed to room temprature. After stirring for 10 hours, it was cooled with ice, saturated ammonium chloride aqueous solution (10 mL) was added, and it was diluted with diethyl ether. 2N Hydrochloric acid was added until the salt, which was deposited in the reactor vessel, was completely melted. Five % sodium hydroxide aqueous solution was added to the organic layer, and the water layer was separated after mixed well by shaking. After this aqueous solution was made acidic by the addition of 2M sulfuric acid, it was extracted with ethyl acetate. After the organic layer was concentrated, a diethyl ether solution of diazomethane was added, the reaction solution was concentrated, and a mixture of chemical compound (23): chemical compound (24)=3:1 (24.8 g, 94.6 mmol, 88%) was obtained by purification by silica gel column chromatography.

Iodine (2.60 g, 10.3 mmol) and acetonitrile (30 mL) were put in a 100 mL eggplant-type flask. The acetonitrile solution (20 mL) of the chemical compound (23) (898 mg, 3.42 mmol) was dropped. After stirring for 1.5 hours at room temperature, sodium thiosulfate aqueous solution and sodium hydrogen carbonate aqueous solution were added, and it was diluted with diethyl ether. The organic layer was washed with saturated salt solution, dried with sulfuric anhydride magnesium, filtrered and concentrated, and a chemical compound (25) (743 mg, 1.99 mmol, 58%) was obtained by purification by silica by gel column chromatography

The chemical compound (25) (1.62 g, 4.32 mmol), ethanol (10 mL) and 15% sodium hydroxide aqueous solution (5.1 mL, 22 mmol) were put in a 50 mL eggplant-type flask. After stirring for 1 hour at room temperature, acetic acid (3.5 mL) was added to make its acidity about pH4-5, and the reaction solution was heated to 60° C. After stirring for 1 hour, saturated sodium hydrogen carbonate aqueous solution was added at room temperature, the reaction was stopped, and it was diluted with diethyl ether. The organic layer was washed with saturated salt solution, dried with sulfuric anhydride magnesium, filtrated and concentrated. Then a chemical compound (26) (1.03 g, 3.90 mmol, 90%) was obtained by purification by silica gel column chromatography.

The chemical compound (26) (3.83 g, 14.5 mmol), 1,2-dichloroethane (7.3 mL), diisopropyl ethylamine (7.60 mL, 43.5 mmol) and chloromethyl methyl ether (2.2 mL, 29.0 mmol) were put in a 50 mL eggplant-type flask After stirring for 16 hours at 40° C., methanol (1 mL) was added at 0° C. and the reaction was stopped. The organic layer was washed with saturated ammonium chloride aqueous solution and saturated salt solution, dried with sulfuric anhydride magnesium, filtered and concentrated, and a chemical compound (27) (4.10 g, 13.3 mmol, 92%) was obtained by purification by silica gel column chromatography.

The chemical compound (27) (3.11 g, 10.1 mmol) and THF(20 mL) were put in a 100 mL eggplant-type flask and cooled to −78° C. Allyl magnesium bromide (0.64 M diethyl ether solution, 15.7 mL, 10.1 mmol) was dropped, and stirred for 15 minutes. Saturated ammonium chloride aqueous solution (5 mL) was added, and the reaction was stopped. It was extracted with ethyl acetate, the organic layer was washed with saturated ammonium chloride aqueous solution and saturated salt solution, dried with sulfuric anhydride magnesium, filtered and concentrated, and a crude compound (28) (3.75 g, 10.7 mmol) was quantitatively obtained.

The crude compound (28) (3.75 g) and THF(10 mL) were put in a 100 mL eggplant-type flask and cooled to 0° C. According to the report of H. C. Brown et al., [Journal of American Chemical Society Vol.81, page 1512, 1959], Disiamylborane (1 M THF solution, 25 mL, 25 mmol) prepared just before addition was dropped and stirred for 1 hour. Fifteen % sodium hydroxide aqueous solution (7.1 mL) and 30% hydrogen peroxide water solution (9.1 mL) were added, and stirred for overnight. It was extracted with ethyl acetate, the organic layer was washed with saturated ammonium chloride aqueous solution and saturated salt solution, dried with sulfuric anhydride magnesium, filtered and concentrated, and diol was obtained. 1,2-Dichloroethane (10 mL) and camphor sulfuric acid (69 mg, 0.30 mmol) were added to this, and stirred for one day. The organic layer was washed with saturated sodium hydrogen carbonate aqueous solution and saturated salt solution, dried with sulfuric anhydride magnesium, filtered and concentrated, and a chemical compound (29) [2.77 g, 7.89 mmol, 78% (3 steps)] was obtained by purification by silica gel column chromatography.

The chemical compound (29) (20.4 g, 58.2 mmol), ethyl acetate (58 mL) and 20% Pd(OH)₂ on carbon (0.933 g, 3 mol %) were put in a 200 mL eggplant-type flask. It was connected to a thick-wall balloon, which was filled with hydrogen, and stirred intensively for 19 hours at room temperature under hydrogen air flow. The reaction solution was filtered with celite, the filtrate was concentrated, and a chemical compound (30) (15.1 g, 58.1 mmol, >99%) was obtained by purification by silica gel column chromatography.

Under the argon atmosphere, CH₂Cl₂(150 mL) and DMSO (2.6 mL, 36 mmol) were put in a 20 mL pear-type flask, and cooled to −78° C. (COCl)₂ (2.4 mL, 27 mmol) was added to the flask, and it was stirred for 20 minutes. Subsequently, the compound (30) (4.68 g, 18.0 mmol) dissolved in CH₂Cl₂ (25 mL) was dropped, and it was further stirred for 30 minutes. It was added Et₃N (10 mL, 72 mmol), and stirred for 1 hour under warming to −60° C. Saturated ammonium chloride aqueous solution was added, and the organic layer was washed with saturated ammonium chloride aqueous solution and saturated salt solution. It was dried with sulfuric anhydride magnesium and the solvents were eliminated. Then a crude aldehyde (31) was obtained by purification by phlorizil column chromatography.

The crude aldehyde (31) (about 18.0 mmol) and toluene (90 mL) were put in a 300 mL eggplant-type flask, and cooled to −78° C. According to the report of W. R. Roush et al. [Journal of American Chemical Society Vol.112, page 6639, 1990], (R,R,Z)-crotylboronate (32) (0.65 M toluene solution 29 mL, 18.9 mmol) which was prepared just before addition was dropped, and stirred for 2 hours. NaBH₄ (150 mg) and ethanol (25 mL) were added, and stirred at room temperature. After 15% sodium hydroxide aqueous solution (10 mL) was added, water was also added until the deposited salt was dissolved, and it was then stirred for 2 days. It was extracted with diethyl ether, the organic layer was washed with saturated salt solution, dried with sulfuric anhydride magnesium, filtered and concentrated, and a crude compound (33) (4.82 g, 15.4 mmol, 85%) was obtained by purification by silica gel column chromatography.

The crude compound (33) (4.82 g, 15.4 mmol) was put in a 1L eggplant-type flask, THF (5 mL) and DMF (5 mL) were added, and it was stirred at 0° C. After NaH (60% suspension in mineral oil: 1.23 g, 31 mmol) was gradually added, benzil bromide (2.8 mL, 23 mmol) was dropped. After stirring for 7 hours at room temperature, the reaction was stopped by addition of methanol, and it was further stirred for overnight. The organic layer was extracted, washed with saturated ammonium chloride aqueous solution and saturated salt solution, dried with sulfuric anhydride magnesium, filtered and concentrated. A chemical compound (34) (3.78 g, 9.36 mmol, 52% (3 steps from 30)) was obtained by purification by silica gel chromatography.

The chemical compound (34) (420 mg, 1.04 mmol), tBuOH (2.5 mL), water (2.5 mL), N-methylmorpholine oxide aqueous solution (50%, 0.65 mL, 3.1 mmol) and osmium tetraoxide (19 μM tBuOH solution, 550 μl, 10.4 μmol) were added in a 20 mL eggplant-type flask, and stirred for 27 hour at room temperature. After adding NaIO₄ (445 mg, 2.08 mmol) and stirring for 1 hour at room temperature; water and saturated sodium hydrogencarbonate aqueous solution were added, it was extracted with diethyl ether, and then the organic layer was washed with saturated salt solution. It was dried with sulfuric anhydride magnesium, the solvents were eliminated, and a crude aldehydes (530 mg) was obtained. The crude aldehyde, tBuOH (8 mL), water (2 mL), NaH₂PO₄.H₂O(600 mg), 2-methyl-2-butene (1.1 mL, 10 mmol) and NaClO₂ (671 mg, 7.42 mmol) were put in a 20 mL eggplant-type flask, and it was stirred for 2 hour at room temperature. It was extracted with ethyl acetate, and the organic layer was washed with saturated salt solution and concentrated. A chemical compound (35) (437 mg, 1.03 mmol, 99%) was obtained by purification by phlorizil column chromatography.

The chemical compound (19) (4.61 g, 7.86 mmol), the chemical compound (35) (2.34 g, 5.55 mmol), EDC.HCl (1.60 g, 8.33 mmol), DMAP (68 mg, 0.56 mmol) and CH₂Cl₂ (5.6 mL) were put in a 30 mL eggplant-type flask. After stirring for 15 hours at 35° C., the solvents were eliminated and a chemical compound (36) (3.28 g, 3.22 mmol, 58%) was obtained by purification by silica gel column chromatography.

MS4A (200 mg: made by Aldrich, produced as a powder-state and activated by heating under a reduced pressure), Cp₂TiCl₂ (496 mg, 2 mmol), Mg (58 mg, 2.4 mmol) and THF (4 mL) were put in a 20 mL eggplant-type flask with a cock, and P(OEt)₃ (0.69 mL, 4.0 mmol) was dropped while stirring at room temperature. After stirring for 2.5 hours at room temprature, it was heated to 50° C., and the chemical compound (36) (504 mg, 0.494 mmol) dissolved in THF (5 mL) was dropped. After 10 minutes, it was heated further and refluxed for 50 minutes. It was cooled to room temperature and 1N NaOH (15 μl) was added. The reaction solution was filtrered and the precipitate was well washed with ether and water. The filtrate was extracted with ether and the organic layer was washed with saturated salt solution, dried with carbon dioxide potassium, filtrered and concentrated. A chemical compound (37) (239 mg, 0.304 mmol, 62%) was obtained by purification by silica gel column chromatography.

Under the argon atmosphere, the chemical compound (37) (1.24 g, 1.58 mmol) and THF (16 mL) were put in a 50 mL eggplant-type flask. After cooling to 0° C., BF₃.THF (1.75 mL, 1.74 mmol) was dropped slowly, and stirred for 50 minutes at room temperature. It was again cooled to −10° C., BF₃.THF (0.16 mL, 0.16 mmol) was dropped, and stirred for 50 minutes at room temperature. It was cooled to −10° C., added 15% NaOH (1.9 mL) and 30% hydrogen peroxide water solution (1.8 mL), and stirred for 12 hour at room temperature. The organic layer was washed with saturated ammonium chloride aqueous solution and saturated salt solution. After drying with sulfuric anhydride magnesium, the solvents were eliminated, and a chemical compound (38) (950 mg, 1.18 mmol, 75%) was obtainedbypurification by silica gel column chromatography.

Under the argon atmosphere, CH₂Cl₂ (10 mL) and DMSO (0.49 mL, 6.9 mmol) were put in a 50 mL pear-type flask, and it was cooled to −78° C. (COCl)₂ (0.37 mL, 4.2 mmol) was added to the flask, and it was stirred for 10 minutes. Subsequently, the chemical compound (38) (1.11 mg, 1.38 mmol) dissolved in CH₂Cl₂ (6.5 mL) was dropped, and it was further stirred for 1 hour and 20 minutes. After adding Et₃N (2.0 mL, 14 mmol), it was warmed slowly for 1 hour and added saturated ammonium chloride aqueous solution at −40° C., and then the reaction was stopped. The organic layer was washed with saturated ammonium chloride aqueous solution and saturated salt solution. After drying with sulfuric anhydride magnesium, the solvents were eliminated, and a mixture of chemical compound (39) and chemical compound (40) was obtained by purification by silica gel column chromatography. By medium-pressure column chromatography (YAMAZEN, Si40B column), an objective chemical compound (39) (346 mg, 0.432 mmol, 31%) and a chemical compound (40) (628 mg, 0.784 mmol, 57%) were separately obtained.

The epi-form compound (40) (628 mg, 0.78 mmol, 57%) was dissolved in CH₂Cl₂ (5 mL) and DBU (0.25 mL) was added. After stirring for 4 hours at room temperature, it was diluted with hexane-ethyl acetate, and the organic layer was washed with saturated ammonium chloride aqueous solution and saturated salt solution. After drying with sulfuric anhydride magnesium and eliminating the solvents, the chemical compound (39) (285 mg) and the chemical compound (40) (326 mg) were separately obtained by medium-pressure column chromatography (Si40B column, YAMAZEN).

The recovered epi-form compound (40) (326 mg) was dissolved in CH₂Cl₂ (3mL), and DBU (0.15 mL) was added. After stirring for 3.5 hours at room temperature, it was diluted with hexane-ethyl acetate, and the organic layer was washed with saturated ammonium chloride aqueous solution and saturated salt solution. After drying with sulfuric anhydride magnesium and eliminating the solvents, the chemical compound (39) (136 mg) and chemical compound (40) (186 mg) were separately obtained by the medium-pressure column chromatography (Si40B column, YAMAZEN).

Again, the recovered epi-form compound (40) (186 mg) was dissolved in CH₂Cl₂ (2 mL), and DBU (0.10 mL) was added. After stirring for 3 hours at room temperature, it was diluted with hexane-ethyl acetate, and the organic layer was washed with saturated ammonium chloride aqueous solution and saturated salt solution. After drying with sulfuric anhydride magnesium and eliminating the solvents, it was separated by the medium-pressure column chromatography (Si40B column, YAMAZEN). By repeating the isomerization 3 times, the chemical compound (39) (367 mg, 458 mmol, 58%) was obtained, and the chemical compound (40) (75.4 mg, 94.1 μmol, 12%) was recovered.

The chemical compound (39) (128 mg, 0.351 mmol), hexane (3.5 mL) and CH(OMe)₃ (0.35 mL) were put in a 20 mL eggplant-type flask, and stirred. TfOH(0.25 M in CH₂Cl₂, 42 μl, 10.5 mmol) was dropped, and stirred for 27 hour at room temperature. The organic layer was washed with saturated sodium hydrogen carbonate aqueous solution and saturated salt solution. After drying with sulfuric anhydride magnesium, the solvents were eliminated, and a diastereomer mixture at position 10 (41) (227 mg, 0.295 mmol, 84%) was obtained by purification by silica gel column chromatography.

The mixture (41) (103.6 mg, 134 μmol), CH₂Cl₂ (1.1 mL) and Et₃SiH (0.48 mL, 2.68 mmol) were put in a 10 mL eggplant-type flask and cooled to −55° C. The CH₂Cl₂ (330 μl) solution of BF₃.Et₂O (33 μl, 0.268 mmol) was dropped, and stirred for 50 minutes. After the reaction solution was diluted by the 1% Et₃N- hexane solution at −15° C., saturated sodium hydrogen carbonate aqueous solution was added. The organic layer was washed with saturated salt solution, dried with sulfuric anhydride magnesium, filtered and concentrated. A chemical compound (42) (70.9 mg, 96 μmol, 71%) was obtained by purification by silica gel column chromatography.

The chemical compound (43) (7.8 mg, 11 μmol), EtOAc (100 μl), MeOH (300 μl) and Pd(OH)₂ (20% on carbon: 1.1 mg, 11 μmol) were put in a 20 mL eggplant-type flask. Under the hydrogen atmosphere (using a thick-wall balloon), it was stirred for 3 hours at room temperature. It was diluted with ethyl acetate and filtrated with celite. The filtrate was concentrated, and a chemical compound (44) (6.2 mg, 13 μmol) was quantitatively obtained.

The chemical compound (44) (4.2 mg, 9.0 μmol), CH₂Cl₂ (300 μl), (MeO)₂CHCH₂CO₂Me (25 μl, 180 μmol) and TsOH.H₂O (0.5 mg, 3 μmol) were put in a 20 mL eggplant-type flask. After stirring for 1.5 hours at room temperature, toluene (1 mL) was added, decompressed (120 mbar/hPa) using a rotary evaporator, and returned to atmospheric pressure after 1 hour. After diluted with ethyl acetate, the organic layer was washed with saturated sodium hydrogen carbonate aqueous solution, water and saturated salt solution, dried with sulfuric anhydride magnesium, filtered and concentrated. A mixture of diastereomers at the acetal position (chemical compound (45a): chemical compound (45b)=3:1 ) (4.7 mg, 8.5 μmol, 94%) was obtained by purification by silica gel column chromatography.

The chemical compounds (45) (4.7 mg, 8.5 μmol), t-BuOH (0.5 mL), water (125 μL) and LiOH.H₂O (2.8 mg, 68 μmol) were added in a 20 mL pear-type flask and stirred at room temperature for 1 hour. After KHSO₄ (18.6 mg, 136 μmol) was added and pH (about 3-4) of the solution was confirmed, it was diluted with ethyl acetate (10 mL). After it was dried with sulfuric anhydride magnesium, filtered and concentrated, and a crude compound (46) was obtained. DMF (200 μL), N- hydroxy succinimide (9.7 mg, 85 μmol) and EDC.HCl (8.1 mg, 43 μmol) were added to this and stirred for 12 hour at room temperature. The reaction solution was diluted with ethyl acetate (10 mL), the organic layers were washed 3 times in water, dried with sulfuric anhydride sodium and concentrated, and an activated ester (47) was obtained. A solution added DMF (100 μL) to it was prepared for preparation of conjugates.

The DMF solution (50 μl) containing the activated ester (47) (about 4.2 μmol) was added to the PBS buffer solution (2.0 mL) containing KLH (7.0 mg) and stirred for 10 minutes. After one day of storage, it was dialyzed at 4° C. After both 14 and 19 hours, the PBS buffer solution (700 mL) was exchanged, it was transferred from the dialysis membrane to the Eppendorf tube and preserved at −78° C.

The DMF solution (50 μl) containing the activated ester (47) (about 4.2 μmol) was added to the PBS buffer solution (2.0 mL) containing BSA (7.0 mg), and stirred for 10 minutes. After storage for 1 day, it was dialyzed at 4° C. After both 14 and 19 hours, the PBS buffer solution (700 mL) was exchanged, it was transferred from the dialysis membrane to the Eppendorf tube and preserved at −78° C.

The BSA conjugate obtained by dialysis was analyzed using MALDI-TOF-MS. The mean molecular weight of the BSA conjugate was about 71,800 (The molecular weight of BSA was 66,400). Since the molecular weight of the hapten is (540), it was found that an average of 10 haptens was coupled in the BSA conjugate.

2. The Preparation of Monoclonal Antibody

After the RIBI adjuvant (RIBI, made by Immunol. Res. Inst.) was added to the IJKLM-KLH (100 μg) conjugate obtained in the preceding experiments and emulsified by stirring well, the emulsion was given intraperitoneally to the Balb/c mice (5 mice) 3 times every 2 weeks. The sera of the mice were collected on day 35 after the first immunization, and the antibody titers of the sera were titrated by the ELISA method using IJKLM-BSA and ABC-BSA.

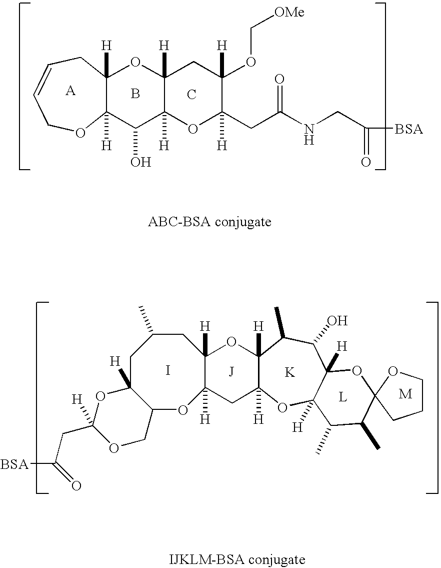

Fifty μl of the hapten-BSA (IJKLM-BSA or ABC-BSA) solution was put into each well of 96-well plates for ELISA (made by The Falcon Co., 3910), and after the plates were left for 2 hours at room temperature, these conjugates were adsorbed to the plates by keeping them at 4° C. for overnight. After the plates were washed 3 times with PBS-Tween [PBS buffer solution containing 5% Tween 20 (Wako Junyaku, Polyoxyethylene (20) Sorbitan Monolaurate made by The ICI Co., Trademark: Tween 20, Compound No. 167-11515)], they were then washed once with the MILLI-Q water, and the un-adsorbed conjugates were removed. The supernatants cultured hybridomas (or antiserum, or purified antibody solution) were added, kept at room temperature for 1 hour, and washed with PBS-Tween and the MILLI-Q water in order. Fifty μl of the enzyme labeled secondary antibody (goat anti-mouse IgG-West horseradish peroxidase) (The ZYMED Co, 62-6520, 1,000 times dilution) was put into each well, and after keeping them at room temperature for 1 hour, they were washed with PBS-Tween and the MILLI-Q water in order. After the addition of 100 μl of a substrate solution [the contents of the substrate solution: 1,2-phenylenediamine 4.0 mg, hydrogen peroxide water 10 μl, 0.1 M citrate buffer solution (pH 5.0) 10 mL] and processing for color reaction for 5 minutes, the reaction was stopped by adding 2 M sulfuric acid (50 μl). The absorbance was measured using the microplate absorbancy measuring device (BIO-RAD, Benchmark 170-6850) at 450 nm, and the positive clones were judged.

Fifty μl of the PBS buffer solution was put in the upper most stage of each 96-well plate for ELISA, which was coated with the hapten-BSA (IJKLM-BSA or ABC-BSA) solution. The antiserum (50 μl) of the mouse, diluted at 200 times, was added to the wells (A1) of the upper most stage, and the two-fold dilution of this solution was done in order (400 to 51,200 times dilution was made in tandems A1˜A8). After the plates were stood at room temperature for 1 hour, the absorbance at 450 nm was measured by the above-mentioned method. A sigmoid titration curve was formed when the logarithm of serum dilution rate and absorbance were plotted.

Table 1 shows the antibody titers of the sera obtained (Numbers 1˜5 on the table represent the mouse number). It was found that the antibody in the sera bound with the IJKLM-BSA conjugate depended on the serum concentration, but did not with the ABC-BSA conjugate.

TABLE 1

| ilution | Titrated with IJKLM-BSA | | | | | Titrated with ABC-BSA | | | | |
|---|---|---|---|---|---|---|---|---|---|---|
| 00 | .829 | .867 | .848 | .846 | .853 | .216 | .275 | .209 | .211 | .220 |
| 00 | .810 | .831 | .819 | .839 | .800 | .142 | .168 | .117 | .126 | .113 |
| 600 | .757 | .733 | .761 | .721 | .745 | .098 | .114 | .087 | .090 | .084 |
| 200 | .734 | .715 | .738 | .755 | .745 | .098 | .082 | .068 | .066 | .063 |
| 400 | .685 | .627 | .669 | .709 | .709 | .056 | .060 | .054 | .058 | .056 |
| 2800 | .595 | .469 | .568 | .664 | .674 | .050 | .052 | .050 | .053 | .053 |
| 5600 | .473 | .345 | .392 | .527 | .494 | .045 | .046 | .044 | .046 | .045 |
| 1200 | .357 | .240 | .275 | .384 | .414 | .044 | .047 | .045 | 052 | .071 |

Among the 5 mice, the mouse showing the highest antibody titer was given a booster by intraperitoneal injection of IJKLM-KLH (100 μg) and its spleen was isolated after 3 days. After the tissues and the fragments attached to the organ were removed using forceps, it was transferred to the Petri dish added a basal medium [RPMI Medium 1640 (made by The GIBCO Co., one bag), 2 g sodium hydrogencarbonate, 20 mg penicillin-streptomycin (made by The GIBCO Co.,), and 20 mL of 200 mM- glutamine dissolved in distilled water to make a 1000 mL solution with pH 7.2], and the cells in the spleen were suspended using forceps. After the splenic cell suspension was filtered, it was transferred to a 50 mL centrifuge tube. Furthermore, 15 mL of the basal medium was added, pipetted well, and filtered to prepare a 30 mL cell suspension. It was centrifuged at 800 rpm for 5 minutes at room temperature, the supernatant was removed and tapped. Thirty mL of the HT.BC medium [a mixture of 200 mL of fetal bovine serum (FCS), 20 mL of HT (the HT solution was made by The Cosmo Bio Co., 50-times concentration), 50 mL of BC (made by The Bioresearch Island Co., BriClone) and 730 mL of basal medium] was added, and the cells were suspended.

The P3X63-Ag8.653 myeloma cells (made by The Dainippon Pharmaceutical) were taken out from the refrigerator (−130° C.), and rapidly thawed in a 37° C. incubator. After the tube was well disinfected with alcohol cotton, the cell suspension in the tube was transferred to 30 mL of the basal medium. It was centrifuged at 800 rpm for 5 minutes at room temperature, and the supernatant was removed. After tapping, 10 mL of 10% FCS medium (10% FCS was added to the basal medium) was added, and the cells were suspended and transferred to a 50 mL culture flask. The plug of the flask was loosened, and it was put in a CO₂ incubator. It was subcultured every 1˜2 days, and divided into two 250 mL flasks (90-100 mL).

The splenic cells (2×10⁸ cells) obtained from the mouse were mixed with myeloma cells (5×10⁷ cells), centrifuged (800 rpm, for 5 minutes, at room temperature), and then the supernatant was removed and tapped. Thereafter, 30 mL of the ECF buffer solution [45.5 g of mannitol, 10 mM calcium chloride (10 mL), 10 mM magnesium chloride (10 mL) and 20 mM tris buffer solution with a pH of 7.2 were dissolved in distilled water to make 1000 mL] was added, centrifuged (800 rpm, for 5 minutes, at room temperature), removed the supernatant, and tapped. These procedures were repeated 2 times, prior to the addition of the ECF buffer solution (4.8 mL). A 1.2 mL aliquot of the solution was pipetted into each well of 6-well plates (made by the SUMIRON), and the cells were fused using the SSH-10 cell fusion equipment made by The Shimadzu Co. by the following specifications [distance between electrodes: 1.0 mm; alternating current frequency: 1 MHz; alternating current initial stage applied voltage: 80V; alternating current, initial stage applied time: 10 s; pulse duration: 40 μs; pulse voltage: 920 V; pulsed electric field strength: 2.30 kV/cm ; secondary applied alternating current voltage: 80 V; applied pulse interval: 1.0 s; applied number of pulse: 1; pulse voltage change: +0 V ; last alternating current applied time: 10 s; AC voltage damping factor 0%; contact strengthening: off].

The hybridoma cell preparation was transferred to each well of 10 plates of a 96-well plate, in which 100 μl of the HAT medium (selection medium) [110 mL basal medium, 30 mL FCS, 7.5 mL BC, HAT (made by The Cosmo Bio Co., HAT liquid (50 times concentration))] was contained. After 2 weeks, hybridomas which produce the antibody that binds to the hapten of the IJKLM-ring fragment, were screened using IJKLM-BSA by the ELISA method. After positive wells were selected and cloned 2 times, the positive monoclones that were confirmed to produce the antibody in the repeated ELISA were cultivated successively to proliferate to produce about 200 mL of the cell suspension. As the results are shown in Table 2, 3 kinds of monoclonal antibodies, were successfully produced using IJKLM-KLH as an immunogen.

3. Binding Test of the Monoclonal Antibodies

The binding test of the monoclonal antibodies obtained was carried out using the ELISA method. The BSA conjugates, which were coupled with the partial structures of ciguatoxins shown below, were used as antigens.

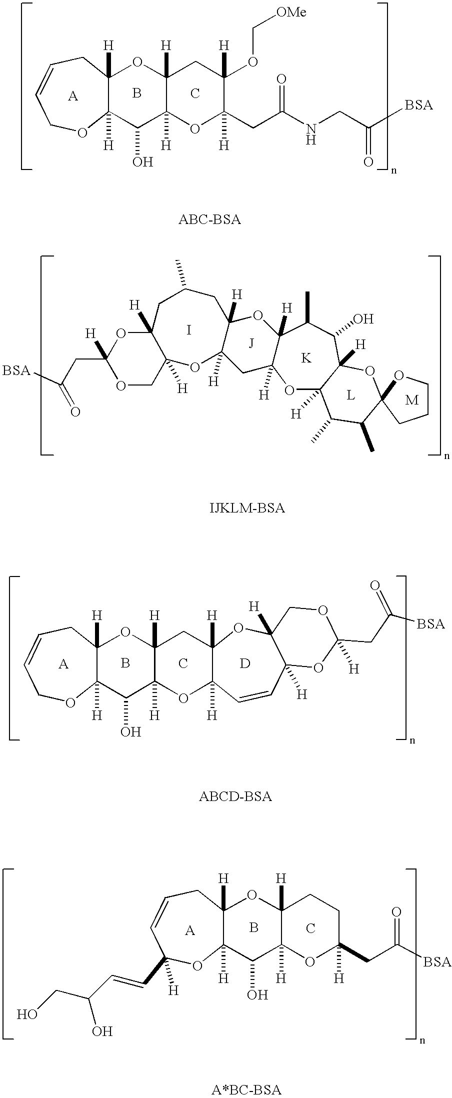

The results are shown in Table 2.

TABLE 2

| Monoclonal | | ABC- | ABCD- | A*BC- | IJKLM- | Kd for |
|---|---|---|---|---|---|---|
| antibody | Isotype | BSA | BSA | BSA | BSA | IJKLM-BSA |
| IM-3D11 | IgG₁ λ | − | − | − | +++ | 8.6 |
| IM-2C7 | IgM κ | ++ | ++ | ++ | ++ | * |
| TM-8B12 | IgM κ | ++ | ++ | ++ | ++ | * |

+++: strong reaction,
++: medium reaction,
+: weak reaction,
−: no reaction, no reaction,
*: not measured

The IM-3D11 cells were selected as the cells which produce the IgG antibody with a high affinity to IJKLM-BSA and a high specificity to the hapten.

The supernatants were purified using the anti-mouse IgG and IgM affinity columns (made by NGF Industries Ltd.) (phosphate buffer for binding (pH 7.0), buffer for elution (0.2 M Glycine-HCl, pH 2.5)). The purified antibody was confirmed to be >95% in purity by the SDS-PAGE analysis. As shown in Table 2, the subclasses of these antibodies were determined using a typing kit (37501) made by PIERCE.

Next, the selected monoclonal antibody was purified, and its dissociation constant (Kd) for hapten was determined. The solution of a serial two-fold dilution of a competitive inhibitor (each 30 μl of PBS solution) was added to the ELISA plates (from the A1 to the A12 well). An antibody solution (30 μl) was added to the wells, and kept at room temperature for 2 hours. Fifty μl of the mixed solution of antibody and inhibitor was added to the 96-well ELISA plates (made by The Falcon Co., 3910) coated with the hapten-BSA solution, and kept at room temperature for 20 minutes. After the plates were washed, the absorbance was measured and a titration curve was obtained. Referring to the method of Friguet et al. [Journal of Immunological Method Vol.77 (1985), page 305], the Kd value of the inhibitor was determined from the gradient of straight line obtained by the Klotz plotting. From the results, the dissociation constant of the coupling of IM-3D11 and the IJKLM-ring fragment (IM)

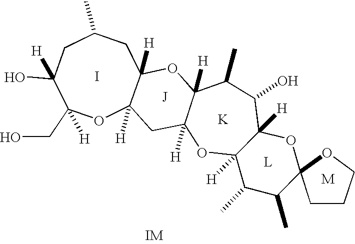

as a hapten was Kd=8.6 nM and IM-3D11 was found to show a considerably high affinity to the IJKLM-ring fragment (Table 2).

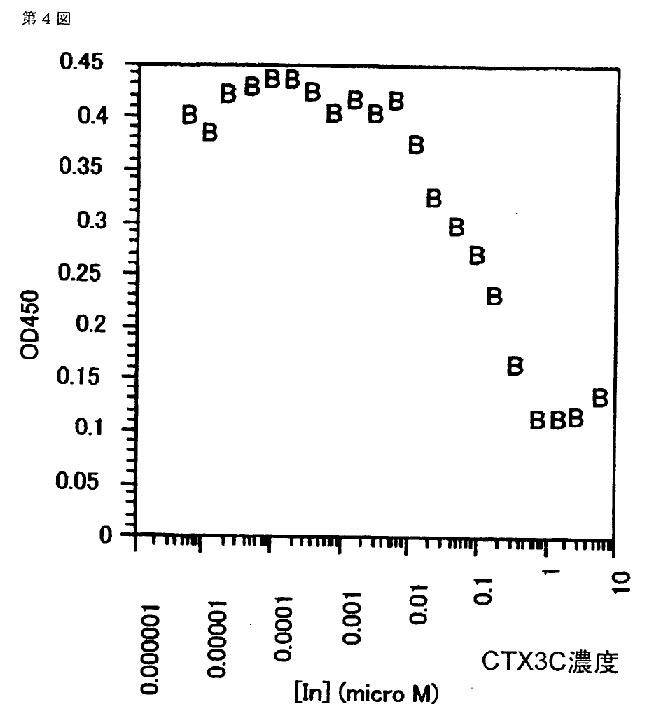

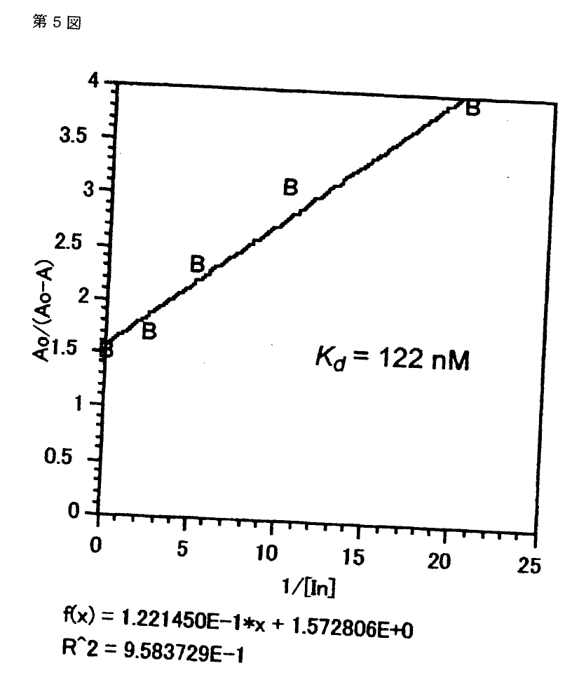

Based on these results, binding with the toxin's main body CTX3C was then examined using the above-mentioned experimental system for competitive inhibition. The results are shown in FIGS. 4 and 5. From the experiment, it was found that IM-3D11 bound strongly to CTX3C (Kd=0.122 μM). In addition, IM-3D11 little bound to okadaic acid (Kd>100 μM) and maitotoxin (Kd>25 μM), when marine polyether toxins with similar chemical structures were examined in the binding assay. The red tide poison brevetoxins showed a cross reactivity, but it was very weak compared to the affinity of CTX3C, the former being about 1/350 of the latter (Brevetoxin A: Kd=43 μM, Brevetoxin B: Kd>100 μM).

Advantageous Effect of the Invention

Up to now, no monoclonal antibody that binds specifically and strongly to ciguatoxins are known. Therefore, it has been difficult to prove whether ciguatoxins were included in the samples tested or not, even if the samples showed a positive result in the assay using the existing antibodies. However, the monoclonal antibody of the present invention, which utilizes the IJKLM-ring fragment of ciguatoxins as a hapten, strongly bind to ciguatoxins with a very high specificity and with almost no cross reactivity to okadaic acid and maitotoxin, which is a problem with the existing antibodies. In addition, it is possible to establish an assay system for ciguatoxins with a high reliability by utilizing this antibody.

What is claimed is:

1. A monoclonal antibody which specifically binds to ciguatoxins.

2. A monoclonal antibody specific to ciguatoxins which specifically binds to a protein conjugate prepared by coupling a synthetic hapten, having a partial structure capable of being an antigenic determinant for ciguatoxins, and a protein.

3. The monoclonal antibody of claim 2, wherein said protein conjugate is represented by the following formula (wherein n is a positive number).

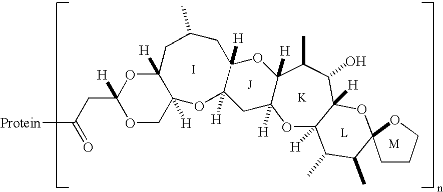

4. The monoclonal antibody of claim 3 wherein said protein is BSA (bovine serum albumin) or KLH (keyhole limpet hemocyanin).

5. A detection reagent for ciguatoxins, comprising the monoclonal antibody of any of claims 1-4 as an active component.

6. A protein conjugate prepared by coupling a synthetic hapten having a partial structure capable of being an antigenic determinant for ciguatoxins and a protein.

7. A protein conjugate represented by the following formula (wherein n is a positive number).

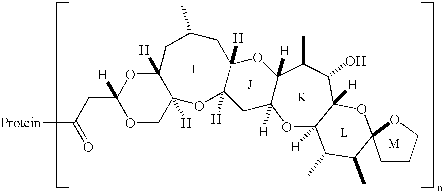

8. The protein conjugate of claim 7 wherein said protein is BSA (bovine serum albumin) or KLH (keyhole limpet hemocyanin).
